SEQUENCE LISTING (5 sequences)

SEQ ID NO: 1 - protein, 1374 aa, Homo sapiens
MSTEADEGITFSVPPFAPSGFCTIPEGGICRRGGAAAVGEGEEHQLPPPPPGSFWNVESAAAPGIGCPAATSSSSATRGRGSSVGGGSRRTTVAYVINEASQGQLVVAESEALQSLREACETVGATLETLHFGKLDFGETTVLDRFYNADIAVVEMSDAFRQPSLFYHLGVRESFSMANNIILYCDTNSDSLQSLKEIICQKNTMCTGNYTFVPYMITPHNKVYCCDSSFMKGLTELMQPNFELLLGPICLPLVDRFIQLLKVAQASSSQYFRESILNDIRKARNLYTGKELAAELARIRQRVDNIEVLTADIVINLLLSYRDIQDYDSIVKLVETLEKLPTFDLASHHHVKFHYAFALNRRNLPGDRAKALDIMIPMVQSEGQVASDMYCLVGRIYKDMFLDSNFTDTESRDHGASWFKKAFESEPTLQSGINYAVLLLAAGHQFESSFELRKVGVKLSSLLGKKGNLEKLQSYWEVGFFLGASVLANDHMRVIQASEKLFKLKTPAWYLKSIVETILIYKHFVKLTTEQPVAKQELVDFWMDFLVEATKTDVTVVRFPVLILEPTKIYQPSYLSINNEVEEKTISIWHVLPDDKKGIHEWNFSASSVRGVSISKFEERCCFLYVLHNSDDFQIYFCTELHCKKFFEMVNTITEEKGRSTEEGDCESDLLEYDYEYDENGDRVVLGKGTYGIVYAGRDLSNQVRIAIKEIPERDSRYSQPLHEEIALHKHLKHKNIVQYLGSFSENGFIKIFMEQVPGGSLSALLRSKWGPLKDNEQTIGFYTKQILEGLKYLHDNQIVHRDIKGDNVLINTYSGVLKISDFGTSKRLAGINPCTETFTGTLQYMAPEIIDKGPRGYGKAADIWSLGCTIIEMATGKPPFYELGEPQAAMFKVGMFKVHPEIPESMSAEAKAFILKCFEPDPDKRACANDLLVDEFLKVSSKKKKTQPKLSALSAGSNEYLRSISLPVPVLVEDTSSSSEYGSVSPDTELKVDPFSFKTRAKSCGERDVKGIRTLFLGIPDENFEDHSAPPSPEEKDSGFFMLRKDSERRATLHRILTEDQDKIVRNLMESLAQGAEEPKLKWEHITTLIASLREFVRSTDRKIIATTLSKLKLELDFDSHGISQVQVVLFGFQDAVNKVLRNHNIKPHWMFALDSIIRKAVQTAITILVPELRPHFSLASESDTADQEDLDVEDDHEEQPSNQTVRRPQAVIEDAVATSGVSTLSSTVSHDSQSAHRSLNVQLGRMKIETNRLLEELVRKEKELQALLHRAIEEKDQEIKHLKLKSQPIEIPELPVFHLNSSGTNTEDSELTDWLRVNGADEDTISRFLAEDYTLLDVLYYVTRDDLKCLRLRGGMLCTLWKAIIDFRNKQT

SEQ ID NO: 2 - protein, 30 aa, Homo sapiens
SDFGTSKRLAGINPCTETFTGTLQYMAPEI

SEQ ID NO: 3 - protein, 270 aa, Homo sapiens
LEYDYEYDENGDRVVLGKGTYGIVYAGRDLSNQVRIAIKEIPERDSRYSQPLHEEIALHKHLKHKNIVQYLGSFSENGFIKIFMEQVPGGSLSALLRSKWGPLKDNEQTIGFYTKQILEGLKYLHDNQIVHRDIKGDNVLINTYSGVLKISDFGTSKRLAGINPCTETFTGTLQYMAPEIIDKGPRGYGKAADIWSLGCTIIEMATGKPPFYELGEPQAAMFKVGMFKVHPEIPESMSAEAKAFILKCFEPDPDKRACANDLLVDEFLKV

SEQ ID NO: 4 - protein, 270 aa, Homo sapiens
VQSEGQVASDMYCLVGRIYKDMFLDSNFTDTESRDHGASWFKKAFESEPTLQSGINYAVLLLAAGHQFESSFELRKVGVKLSSLLGKKGNLEKLQSYWEVGFFLGASVLANDHMRVIQASEKLFKLKTPAWYLKSIVETILIYKHFVKLTTEQPVAKQELVDFWMDFLVEATKTDVTVVRFPVLILEPTKIYQPSYLSINNEVEEKTISIWHVLPDDKKGIHEWNFSASSVRGVSISKFEERCCFLYVLHNSDDFQIYFCTELHCKKFFE

SEQ ID NO: 5 - protein, 27 aa, Homo sapiens
DFGISGYLVDSVAKTMDAGCKPYMAPE

(19) United States
(12) Patent Application Publication
(10) Pub. No.: US 2013/0236441 A1
(43) Pub. Date: Sep. 12, 2013

(54) METHODS OF TREATING AND PREVENTING THROMBOTIC DISEASES USING ASK1 INHIBITORS

(71) Applicants: Ulhas P. Naik, Hockessin, DE (US); Meghna U. Naik, Hockessin, DE (US)

(72) Inventors: Ulhas P. Naik, Hockessin, DE (US); Meghna U. Naik, Hockessin, DE (US)

(73) Assignee: University of Delaware, Newark, DE (US)

(21) Appl. No.: 13/988,127

(22) PCT Filed: Nov. 18, 2011
(86) PCT No.: PCT/US11/61399

§ 371 Date: May 17, 2013

Related U.S. Application Data
(60) Provisional application No. 61/415,078, filed on Nov. 18, 2010.

(51) Int. Cl. A61K 38/46; A61K 38/45; C12Q 1/48; A61K 38/00

(52) U.S. Cl. CPC A61K 38/465; A61K 38/005; A61K 38/45; C12Q 1/485
USPC 424/94.5; 514/14.9; 424/94.6; 506/9

(57) ABSTRACT

A method of treating or preventing a thrombotic disease in a subject in need thereof comprises administering to the subject an effective amount of a pharmaceutical composition comprising an inhibitor of an apoptosis signal regulating kinase 1 (ASK1) protein. A method of identifying an inhibitor of an apoptosis signal regulating kinase 1 (ASK1) protein useful for treating or preventing a thrombotic disease, comprising (a) contacting a candidate agent with a test sample comprising the ASK1 protein, and (b) comparing the ASK1 protein activity in the test sample with the ASK1 protein activity in a control sample that has not been contacted with the candidate agent, whereby a decrease in the ASK1 protein activity in the test sample compared with the control sample indicates that the candidate agent is an ASK1 inhibitor.

### CROSS-REFERENCE TO RELATED APPLICATION

This application claims the benefit of U.S. Provisional Application No. 61/415,078, filed Nov. 18, 2010, the contents of which are incorporated herein in their entireties for all purposes.

### REFERENCE TO U.S. GOVERNMENT SUPPORT

This invention was made with government support under a grant from the National Heart, Lung, and Blood Institute (NHLBI) (Grant No. 2R01-HL57630-10). The United States government has certain rights in the invention.

### FIELD OF THE INVENTION

The invention relates generally to methods of treating or preventing thrombotic diseases. In particular, the invention relates to the use of apoptosis signal regulating kinase 1 (ASK1) inhibitors to treat or prevent thrombotic diseases.

### BACKGROUND OF THE INVENTION

Blood platelets with unique cell surface receptors play an important role in achieving hemostasis. Unwanted platelet activation, however, results in thrombosis that not only causes complications during surgery, pregnancy and cancer, but also is the main initiator of life-threatening pathological conditions, such as myocardial infarction and stroke, which are leading causes of death worldwide. The identification and functional elucidation of regulators of platelet function may define new targets for developing potential therapeutic agents towards thrombotic disorders. Although significant progress has been made in prevention and treatment, the currently available pharmacological inhibitors, such as P2Y12 and PAR1 antagonists, have limitations. GPIIb/IIIa (integrin α_{IIb}β₃) inhibitors, although effective in inhibiting thrombosis, have severe bleeding complications. The most promising treatment currently available is the use of a combination therapy, such as aspirin and clopidogrel. Aspirin, the most popular and widely used inhibitor of cyclooxygenase, eventually suffers from the development of resistance. Several thrombotic drugs are not very effective and have the side effect of bleeding.

There remains a need for effective anti-thrombotic drugs without much bleeding or other side effects.

### SUMMARY OF THE INVENTION

The present invention relates to methods for treating or preventing a thrombotic disease using an inhibitor of an apoptosis signal regulating kinase 1 (ASK1) protein, and related medicaments and compositions.

A method of treating or preventing a thrombotic disease in a subject in need thereof is provided. The method comprises administering to the subject an effective amount of a pharmaceutical composition comprising an inhibitor of an apoptosis signal regulating kinase 1 (ASK1) protein. The pharmaceutical composition may further comprise a pharmaceutically acceptable carrier or diluent. The pharmaceutical composition may have a pH of 5.0-10.0.

The thrombotic disease may be selected from the group consisting of, but not limited to venous thrombosis, arterial thrombosis, atherosclerosis, arthritis, coagulopathy, deep venous thrombosis (DVT), disseminated intravascular coagulopathy (DIC), pulmonary thromboembolism, Budd-Chiari syndrome, Paget-Schroetter diseases, stroke and myocardial infraction. Other thrombotic complications, which occur following surgery or trauma, could also be treated or prevented in accordance with the invention.

The ASK1 protein may be obtained from activated platelets. The activated platelets may be obtained from a subject who has suffered from the thrombotic disease.

The ASK1 protein may comprise an amino acid sequence of a full-length human ASK1 protein (SEQ ID NO: 1). The ASK1 inhibitor may be capable of attenuating phosphorylation of threonine 838 (T838) SEQ ID NO: 1. The ASK1 inhibitor may be selected from the group consisting of calcium- and integrin-binding protein 1 (CIB1), PP5, 14-3-3ζ, AKT and fragments thereof.

A method of identifying an inhibitor of an apoptosis signal regulating kinase 1 (ASK1) protein useful for treating or preventing a thrombotic disease is also provided. The method comprises (a) contacting a candidate agent with a test sample comprising the ASK1 protein, and (b) comparing the ASK1 protein activity in the test sample with the ASK1 protein activity in a control sample that has not been contacted with the candidate agent. A decrease in the ASK1 protein activity in the test sample compared with the control sample indicates that the candidate agent is an ASK1 inhibitor. The ASK1 protein may be obtained from activated platelets. The activated platelets may be obtained from a subject who has suffered from the thrombotic disease. The thrombotic disease may be venous thrombosis, arterial thrombosis, atherosclerosis, arthritis, coagulopathy, deep venous thrombosis (DVT), disseminated intravascular coagulopathy (DIC), pulmonary thromboembolism, Budd-Chiari syndrome, Paget-Schroetter diseases, stroke or myocardial infraction. Where the ASK1 protein comprises an amino acid sequence of a full-length human ASK1 protein (SEQ ID NO: 1), phosphorylation of threonine 838 (T838) SEQ ID NO: 1 may be attenuated in the test sample compared with the control sample. The identified ASK1 inhibitor may be used in the method of treating or preventing a thrombotic disease according to the present invention.

A medicament is further provided. The medicament comprises an effective amount of an inhibitor of an apoptosis signal regulating kinase 1 (ASK1) protein. The ASK1 inhibitor is useful for treating or preventing a thrombotic disease in a subject. Examples of the thrombotic disease include venous thrombosis, arterial thrombosis, atherosclerosis, arthritis, coagulopathy, deep venous thrombosis (DVT), disseminated intravascular coagulopathy (DIC), pulmonary thromboembolism, Budd-Chiari syndrome, Paget-Schroetter diseases, stroke and myocardial infraction. The ASK1 protein may be obtained from activated platelets. The activated platelets may be obtained from a subject who has suffered from the thrombotic disease.

Where the ASK1 protein comprises an amino acid sequence of a full-length human ASK1 protein (SEQ ID NO: 1), the ASK1 inhibitor in the medicament may be capable of attenuating phosphorylation of threonine 838 (T838) SEQ ID NO: 1. The ASK1 inhibitor may be selected from the group consisting of calcium- and integrin-binding protein 1 (CIB1), PPS, 14-3-3ζ, AKT and fragments thereof. The ASK1 inhibitor may have been identified in accordance with the present invention.

The medicament may comprise a pharmaceutically acceptable carrier or diluent. The medicament may have a pH of 5.0-10.0.

A pharmaceutical composition for treating or preventing a thrombotic disease in a subject is further provided. The pharmaceutical composition comprises an effective amount of an inhibitor of an apoptosis signal regulating kinase 1 (ASK1) protein. The thrombotic disease may be venous thrombosis, arterial thrombosis, atherosclerosis, arthritis, coagulopathy, deep venous thrombosis (DVT), disseminated intravascular coagulopathy (DIC), pulmonary thromboembolism, Budd-Chiari syndrome, Paget-Schroetter diseases, stroke or myocardial infraction. The ASK1 protein may be obtained from activated platelets. The activated platelets may be obtained from a subject who has suffered from the thrombotic disease.

Where the ASK1 protein comprises an amino acid sequence of a full-length human ASK1 protein (SEQ ID NO: 1), the ASK1 inhibitor in the pharmaceutical composition may be capable of attenuating phosphorylation of threonine 838 (T838) SEQ ID NO: 1. The ASK1 inhibitor may be selected from the group consisting of calcium- and integrin-binding protein 1 (CIB1), PP5, 14-3-3ζ, AKT and fragments thereof. The ASK1 inhibitor may have been identified in accordance with the present invention.

The pharmaceutical composition may further comprise a pharmaceutically acceptable carrier or diluent. The pharmaceutical composition may have a pH of 5.0-10.0.

A method of preparing a medicament useful for treating or preventing a thrombotic disease in a subject is provided. The method comprises admixing an inhibitor of an apoptosis signal regulating kinase 1 (ASK1) protein with a pharmaceutically acceptable carrier or diluent. The thrombotic disease may be selected from the group consisting of venous thrombosis, arterial thrombosis, atherosclerosis, arthritis, coagulopathy, deep venous thrombosis (DVT), disseminated intravascular coagulopathy (DIC), pulmonary thromboembolism, Budd-Chiari syndrome, Paget-Schroetter diseases, stroke and myocardial infraction. The ASK1 protein may be obtained from activated platelets. The activated platelets may be obtained from a subject who has suffered from the thrombotic disease. The ASK1 protein may comprise an amino acid sequence of a full-length human ASK1 protein (SEQ ID NO: 1), and the ASK1 inhibitor may be capable of attenuating phosphorylation of threonine 838 (T838) SEQ ID NO: 1. The ASK1 inhibitor may be selected from the group consisting of calcium- and integrin-binding protein 1 (CIB1), PP5, 14-3-3ζ, AKT and fragments thereof. The ASK1 inhibitor may have been identified in accordance with the present invention. The medicament may have a pH of 5.0-10.0.

### BRIEF DESCRIPTION OF THE DRAWINGS

FIG. 1 shows (A) full length human ASK1 protein sequence (SEQ ID NO: 1) and (B) a schematic representation of the linear domain structure of a full length human ASK1, including thioredoxin (TRX) binding domain, N-terminal coil-coil domain (NCC), TNF receptor-associated factors 2/6 (TRAF2/6) (TRAF) binding domain, kinase domain, and C-terminal coil-coil domain (CCC).

FIG. 2 shows expression of an ASK1 protein in both human and mouse platelet lysates by Western blot analysis using anti-ASK1 antibody (Cell Signaling Technology, Inc.).

FIG. 3 shows agonist-induced ASK1 phosphorylation. Washed human platelets (2×10⁸) were stimulated with thrombin (1 U/ml) for various time periods as indicated, and the lysates were Western blotted using phospho-specific antibodies against (A) ASK1, (B) MKK3 and P38, and (C) MKK4, and reprobed with corresponding antibodies against total proteins (Cell Signaling Technology, Inc.) to ensure relatively equal loading of ASK1, MKK3, P38 or MKK4 protein in each of its respective lanes.

FIG. 4 shows tail bleeding time of Ask1^{-/-} mice and Ask1^{+/+} mice after a terminal 3 mm segment of the tail of each anesthetized mouse was amputated and immersed into a warm saline solution. Bleeding time was measured as the time from the start of bleeding to cessation of bleeding.

FIG. 5 shows blood flow in right common carotid artery of anesthetized Ask1^{+/+} mouse (left panel) and Ask1^{-/-} mouse (right panel) monitored for 45 min after an FeCl₃-induced injury. The time for stable thrombotic occlusion (defined as lack of detectable blood flow) after the initiation of arterial injury was recorded.

FIG. 6 shows survival rates (%) of Ask1^{+/+} and Ask1^{-/-} mice from occlusive pulmonary thromboembolism after injection of a mixture of collagen and epinephrine through the tail vein of the anaesthetized mice.

FIG. 7 shows blunted granular release in Ask1 null platelets. (A) ¹⁴C-serotonin-labeled platelets (2.5×10⁸ per ml) from Ask1^{+/+} and Ask1^{-/-} mice were stimulated with various concentration of thrombin for 5 min, and the ¹⁴C-serotonin release was determined using a liquid scintillation counter. (B) Platelets from Ask1^{+/+} and Ask1^{-/-} mice were stimulated with various concentrations of thrombin for 5 min and P-selectin exposure on the platelet surface was determined using flow cytometry.

FIG. 8 shows blunted fibrinogen binding to agonist-activated platelets in the absence of Ask1. Mean fluorescence intensity of FITC-Fg-binding to washed platelets (6×10⁷ per ml) isolated from Ask1^{+/+} and Ask1^{-/-} mice after being stimulated with AYPGKF was quantified by flow cytometry.

FIG. 9 shows impaired clot retraction in Ask1 null platelets. Retraction of clot formed by addition of thrombin (1 U/ml) to a suspension of platelets (3×10⁸ per ml) isolated from an Ask1^{+/+} or Ask1^{-/-} mouse in the presence of fibrinogen was monitored at 37° C. Images of clot retraction at time 0 and after 6 h are shown.

FIG. 10 shows phosphatidylserine (PS) exposure in platelets isolated from Ask1^{+/+} mouse or Ask1^{-/-} mouse. Mean fluorescence intensity of annexin V binding to the washed platelets, which had or had not been stimulated with thrombin, was quantified by flow cytometry.

FIG. 11 shows association of ASK1 with CIB1 or TRAF6 upon activation of human platelets. Unstimulated (un) or thrombin stimulated (ac) platelets (3×10⁸ per ml) lysates were immunoprecipitated with (A) an anti-CIB1 antibody or (B) an anti-ASK1 antibody. IgG was used as a control. (A) The CIB1 immunoprecipitates (IP: CIB1) were Western blotted using an anti-ASK1 antibody and the blot was reprobed with an anti-CIB1 antibody to ensure a relatively equal amount of CIB1 protein in each CIB1 immunoprecipitate. (B) The ASK1 immunoprecipitates (IP: ASK1) were Western blotted using an anti-TRAF6 antibody, and the blot was reprobed with an anti-ASK1 antibody to ensure a relatively equal amount of ASK1 protein in each ASK1 immunoprecipitate. Antibodies were purchased from Cell Signaling Technology, Inc. and Santa Cruz Biotechnology Inc.

FIG. 12 shows phosphorylation of ASK1 Thr845 in platelet lysates upon exposure to LDL or oxidized LDL (oxi-LDL) at 1, 3, 5 and 7 min by Western blot analysis with an anti-phospho-ASK1 antibody.

FIG. 13 is a diagram illustrating the principle of a screening procedure to be used to identify and/or obtain small molecular inhibitors of ASK1 according to some embodiments of the present invention. Phosphorylated ASK1 substrate is detected using sandwiching antibodies. One antibody is directed against phospho-epitope on the p38 or MEK3/6, while the other antibody is directed against another, non-phosphorylated, epitope on a distal part of the substrate. Platelets are treated with thrombin or AYPGKF known to trigger phosphorylation of the substrate and then lysed. The lysate is then mixed with the antibodies, the AlphaScreen Donor beads, and the AlphaScreen Acceptor beads. If the substrate is phosphorylated, the Donor and Acceptor beads will be brought together. Upon laser excitation at 680 nm, the Donor bead will transfer energy to the Acceptor bead if sufficiently close, resulting in the emission of light at 520-620 nm. ASK1 substrate phosphorylation is detected by an increase in the signal at 520-620 nm.

### DETAILED DESCRIPTION OF THE INVENTION

The present invention is based on the discovery that apoptosis signal regulating kinase 1 (ASK1) is expressed in platelets. In particular, ASK1 is dynamically activated during platelet activation, and the absence of ASK1 in a mouse greatly protects the mouse from experimental thrombosis without causing bleeding.

Under physiological conditions, mature platelets are discoid in shape with an invaginated surface and an extensive cytoskeletal meshwork of actin filaments. Upon vascular injury, the monolayer of endothelial cells that line blood vessel wall becomes disrupted, thereby exposing subendothelial adhesive proteins such as collagen and von Willebrand factor (vWF). When circulating platelets attach to these exposed subendothelial matrix proteins, they spread to form a monolayer; however, this is not sufficient to completely seal the vascular wound. The attached platelets secrete their granular contents, including ADP, into the circulation, synthesize arachidonic acid, which is metabolized to thromboxane A₂ (TxA2), a potent platelet agonist, and also generate thrombin on their surfaces. These agonists in turn activate and recruit circulating platelets to the site of injury. As a result of agonist binding to respective receptors, a complex process of signaling events is induced within the platelet that leads to their activation. During platelet stimulation, intracellular calcium levels rise, which profoundly affects the cytoskeleton, causing it to reorganize. As a result, platelets change their shape and a cascade of signaling events are initiated that further induce TxA2 generation, granular secretion, and phosphatidylserine exposure, providing a procoagulant surface to generate thrombin. This signaling cascade ultimately leads to the activation of integrin α_{IIb}β₃, enabling it to bind soluble fibrinogen (Fg). Because Fg is a divalent molecule, it is capable of binding to more than one activated receptor and can thus crosslink platelets to form platelet aggregates. Signaling through ligand-bound integrin is necessary for stabilization of platelet aggregates within a platelet plug. The intact cytoskeleton provides a contractile force for fibrin clot retraction, which facilitates wound healing.

Mitogen-activated protein (MAP) kinases control major cellular responses in organisms contributing to proliferation, migration, differentiation and apoptosis. In humans, at least six subfamilies have been identified. Of these extracellular signal-regulated kinases (ERK1 and 2), c-Jun N-terminal kinases (JNKs) and p38 MAP kinases (p38) have been extensively studied. Growth factors preferentially activate ERK1/2, whereas JNKs and p38s are stimulated by stress stimuli. Each MAP kinase pathway contains a three-tiered kinase cascade comprising a MAP kinase kinase kinase (MAPKKK), a MAP kinase kinase (MAPKK), and a MAP kinase (MAPK). Frequently, a MAPKKK kinase (MAPKKKK) activates MAPKKK. MAPKKKKs are often linked to the plasma membranes through association with a small GTPase of Ras or Rho family or lipid. Human platelets express ERK1, ERK2, JNK1, JNK2, and p38 isozymes (a, b, g, and d). Surprisingly little attention has been paid to the role of MAPKs in hemostasis and thrombosis. ERK2 is phosphorylated and activated by thrombin, collagen, vWF, and ADP. ERK2 activation is independent of Raf-1 and B-Raf, but is dependent on PKCd and MAPKK1/2. JNK1/2 are activated by thrombin, vWF, and ADP. Collagen activates JNK via P2Y12 receptor. p38 is activated by thrombin, TxA2, collagen, vWF, and ADP. It has been shown that patients expressing β₃ integrin pro33 polymorphism associated with increased risk of cardiovascular disease had increased levels of activated MAP kinases and enhanced aggregation response to low doses of agonists. Studies have also showed that physiological platelet agonists induce a rapid, but transient, wave of MAP kinase activation that is necessary for platelet activation by a low dose of agonists. MAP kinases have also been shown to be involved in outside-in integrin signaling. Heterozygous p38 knockout (KO) mice show delayed vessel occlusion times induced by FeCl₃ injury. JNK1 KO mice also have prolonged bleeding times and arteriolar thrombosis occlusion times. Furthermore, platelets from both p38 heterozygous and JNK1 KO mice are associated with blunted aggregation responses to a low dose of agonists. Interestingly, a pan-JNK inhibitor blocked aggregation of JNK1 KO platelets, suggesting that both JNK1 and JNK2 are involved in platelet function. Consistent with this, it has been shown that pan-JNK inhibitor also blocked OxLDL-induced platelet aggregation and abrogated prothrombotic phenotype in high fat diet-fed ApoE null mice.

Apoptosis signal regulating kinase 1 (ASK1) is a serine-threonine kinase that was initially identified as a MAP kinase kinase kinase 5 (MAPKKK5). ASK1 activates MAPKK3, MAPKK4, MAPKK6, and MAPKK7. MAPKK4 and MAPKK7 in turn activate JNK pathway and MAPKK3 and MAPKK6 activate p38 signaling pathway. ASK1 is thus able to activate both JNK and p38 pathways. ASK1 plays a role in apoptosis induced by a variety of cellular stressors including oxidative stress, tumor necrosis factor (TNF)-α, endoplasmic reticulum stress, and anticancer drugs. ASK1-mediated signaling is modulated either positively or negatively by various ASK1 binding proteins, including thioredoxin (TRX), calcium- and integrin-binding protein 1 (CIB1), and TNF receptor-associated factors 2/6 (TRAF2/6).

In resting nucleated cells, it has been shown that ASK1 is present as a homodimer associated through its C-terminal coil-coil domain and each monomer is bound to a reduced thioredoxin (TRX) at the N-terminus. A subpopulation of ASK1 is also known to bind to calcium- and integrin-binding protein 1 (CIB1). CIB1-bound ASK1 is inactive. During cellular activation by a variety of stress stimuli such as ROS or ER stress due to Ca²⁺ release, thioredoxin is oxidized and dissociates from the ASK1 duplex. CIB1 is also shown to dissociate from ASK1 upon Ca²⁺ rise. TRAF2/6 now binds proximal to the kinase domain and the two monomers form a tight association through their N-terminal coil-coil domain. An unknown upstream kinase phosphorylates ASK1 on T845 and renders it active. Activated ASK1 is rapidly deactivated by dephosphorylation of T845 by phosphoprotein phosphatase 5 (PP5), a serine-threonine phosphatase. ASK1 is also phosphorylated on S967 by PDK1. This phosphorylation allows ASK1 to associate with 14-3-g and inhibits ASK1 function. Furthermore, ASK1 is also phosphorylated by AKT on S83, which also renders it inactive.

The terms “protein” and “polypeptide” are used herein interchangeably, and refer to a polymer of amino acid residues with no limitation with respect to the minimum length of the polymer. Preferably, the protein or polypeptide has at least 20 amino acids. The definition includes full-length proteins and fragments thereof, as well as modifications thereof (e.g., glycosylation, phosphorylation, deletions, additions and substitutions).

The term “variant” of a protein used herein refers to a polypeptide having an amino acid or nucleic acid sequence that is the same as the amino acid or nucleic acid sequence of the protein except having at least one amino acid modified, for example, deleted, inserted, or replaced, respectively. A variant of a protein may have an amino acid sequence at least about 80%, 90%, 95%, or 99%, preferably at least about 90%, more preferably at least about 95%, identical to the amino acid sequence or nucleic acid of the protein.

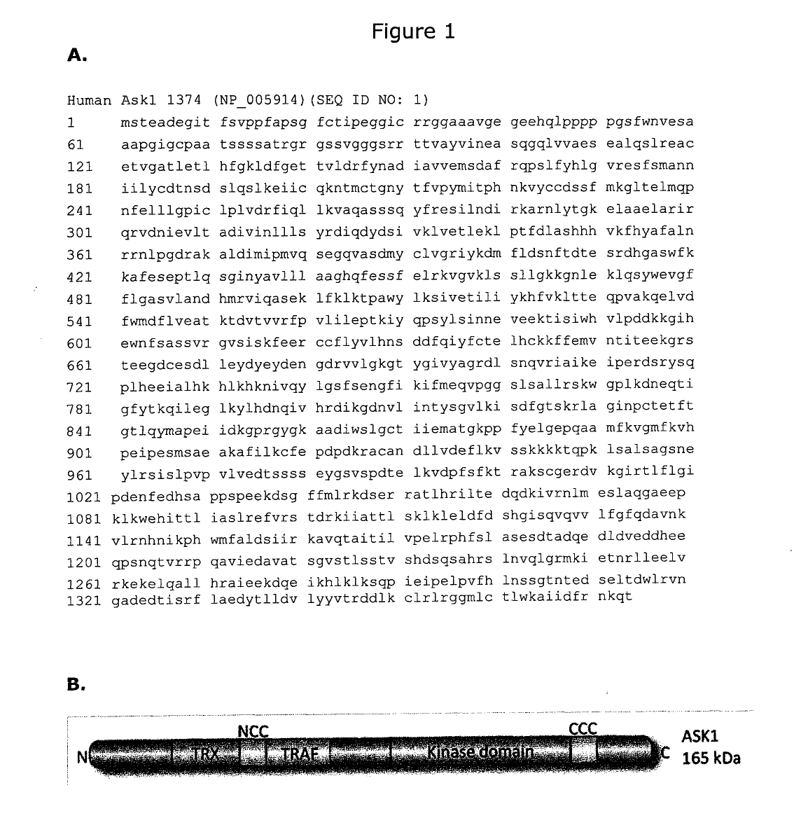

The term “apoptosis signal regulating kinase 1 (ASK1) protein” used herein refers to a full length ASK1 protein, or a functional fragment or variant thereof. The ASK1 protein may be a natural protein or a recombinant protein. A natural ASK1 protein may be obtained from a biological sample, for example, a blood sample comprising platelets. A recombinant ASK1 protein may be obtained using conventional techniques. Full length ASK1 protein sequences and gene sequences in various species are known in the art. For example, the full-length human ASK1 amino acid sequence can be found in the GenBank database Accession No. NP_{—}005914. Human full-length ASK1 protein is a 165 kDa protein having 1374 amino acids (SEQ ID NO: 1) (FIG. 1A), including an active loop at residues 821-850 (SEQ ID NO: 2), a catalytic domain at residues 671-940 (SEQ ID NO: 3), and a CIB1 binding domain at residues 379-648 (SEQ ID NO: 4). The ASK1 substrate sequence may be DFGISGYLVDSVAKTMDAGCKPYMAPE (SEQ ID NO: 5) or a similar sequence. ASK1 has several other functional domains such as N-terminal thioredoxin (TRX) binding domain, kinase domain, NCC domain, CCC domain and a TNF receptor-associated factors 2/6 (TRAF2/6) (TRAF) binding domain (FIG. 1B). A fragment of an ASK1 protein may comprise one or more functional domains (e.g., kinase domain, TRX binding domain, TRAF binding domain, and/or CIB1 binding domain). An ASK1 fragment is preferably a functional fragment. For example, the functional ASK1 fragment may retain the ASK1 kinase activity, capable of phosphorylating a serine and/or threonine residue in a substrate protein comprising the sequence of SEQ ID NO: 5 (i.e., DFGISGYLVDSVAKTMDAGCKPYMAPE) or a similar sequence. A functional ASK1 fragment may comprise the TRAF2/6 binding domain sequence.

The present invention provides a method of treating or preventing a thrombotic disease in a subject in need thereof. The method comprises administering to the subject an effective amount of a pharmaceutical composition comprising an inhibitor of an apoptosis signal regulating kinase 1 (ASK1) protein. Bleeding associated with the thrombotic disease in the subject may be attenuated. For example, the bleeding time may be shortened.

A thrombotic disease may be any disease or disorder associated with the formation of a blood clot in a blood vessel, which may result in reduction of blood flow. Examples of thrombotic diseases include venous thrombosis, arterial thrombosis, atherosclerosis, arthritis, coagulopathy, deep venous thrombosis (DVT), disseminated intravascular coagulopathy (DIC), pulmonary thromboembolism, Budd-Chiari syndrome, Paget-Schroetter diseases, stroke and myocardial infraction. Other thrombotic complications, which occur following surgery or trauma, could also be treated or prevented in accordance with the invention.

A subject may be a mammal, for example, human, horse, cattle (bovine), pig, sheep, goat, dog, and other domestic animals. Preferably, the subject is a human. More preferably, the subject is a human having suffered from or who is predisposed to a thrombotic disease. Most preferably, the subject is a patient who has suffered from a thrombotic disease.

An ASK1 protein may be natural or recombinant. It may be obtained from activated platelets. Platelets may be activated by various agents or agonists, for example, thrombin, collagen, thromboxane A₂ (TxA2), ADP, epinephrine, and platelet activating factor (PAF). Platelets may be activated in vitro or in vivo. Preferably, the activated platelets may be obtained from a subject who has suffered from a thrombotic disease.

An ASK1 inhibitor may be any agent that is capable of inactivating an ASK1 protein. The agent may be a chemical compound or biological molecule (e.g., a protein or antibody). The ASK1 protein activity may be measured by several different methods. For example, the activity of an ASK1 protein may be determined based on the ability of the ASK1 protein to phosphorylate a substrate protein. The substrate protein may comprise an amino acid sequence of SEQ ID NO: 5. Exemplary ASK1 substrate proteins include MAPKK3, MAPKK4, MAPKK6, MAPKK7, or fragments thereof. The ASK1 protein activity may also be measured by the phosphorylation level of the ASK1 protein, for example, the phosphorylation level of a threonine residue in the ASK1 protein corresponding to threonine 838 (T838) of a human full-length ASK1 protein or threonine 845 (T845) of a mouse full-length ASK1 protein. For example, where the ASK1 protein comprises a full-length human ASK1 protein sequence (SEQ ID NO:1), an ASK1 inhibitor may attenuate phosphorylation of T838 in SEQ ID NO: 1. A site specific antibody against human ASK1 T838 or mouse ASK1 T845 may be used to detect the phosphohorylation level.

Where the ASK1 protein comprises an amino acid sequence corresponding to the full-length human ASK1 protein (SEQ ID NO: 1) or the full-length mouse ASK1 protein, the ASK1 inhibitor in the pharmaceutical composition may be capable of attenuating phosphorylation of the threonine residue corresponding to threonine 838 (T838) in the human full-length ASK1 protein or threonine 845 (T845) in the mouse full-length ASK1 protein, respectively. For example, where the ASK1 protein comprises a full-length human ASK1 protein sequence (SEQ ID NO:1), an ASK1 inhibitor in the pharmaceutical composition may attenuate phosphorylation of T838 in SEQ ID NO: 1.

Examples of an ASK1 inhibitor may be selected from the group consisting of calcium- and integrin-binding protein 1 (CIB1), PP5, 14-3-3ζ, AKT and fragments thereof. The protein and gene sequences of CIB1, PP5, 14-3-3ζ, and AKT are known in the art. ASK1 inhibitor fragments preferably retain the inhibitory effect on ASK1. A full length human CIB1 protein contains 199 amino acids (GenBank Accession Number CAG33236.1). A CIB1 fragment may comprise amino acid residues 1-100. Preferably, a CIB1 fragment is capable of binding an ASK1 protein.

The ASK1 inhibitor in the pharmaceutical composition may be selected from the group consisting of calcium- and integrin-binding protein 1 (CIB1), PP5, 14-3-3ζ, AKT and fragments thereof. Preferably, the ASK1 inhibitor is CIB1 or a fragment thereof. More preferably, the ASK1 inhibitor is a full-length human CIB1 protein or a fragment thereof comprising amino acid residues 1-100. The ASK1 inhibitor may have been identified in accordance with the identifying method of the present invention.

The term “an effective amount” refers to an amount of a pharmaceutical composition comprising an ASK1 inhibitor required to achieve a stated goal (e.g., treating or preventing a thrombotic disease in a subject in need thereof). The effective amount of the pharmaceutical composition comprising an ASK1 inhibitor may vary depending upon the stated goals, the physical characteristics of the subject, the nature and severity of the thrombotic disease, the existence of related or unrelated medical conditions, the nature of the ASK1 inhibitor, the composition comprising the ASK1 inhibitor, the means of administering the composition to the subject, and the administration route. A specific dose of an ASK1 inhibitor for a given subject may generally be set by the judgment of a physician. The pharmaceutical composition may be administered to the subject in one or multiple doses. Each dose may comprise an ASK1 inhibitor at about 0.01-5000 mg/kg, preferably about 0.1-1000 mg/kg, more preferably about 1-500 mg/kg.

The pharmaceutical composition may comprise an ASK1 inhibitor in an effective amount for treating or preventing a thrombotic disease in a subject. The pharmaceutical composition may comprise about 0.01-20,000 μg, preferably about 0.1-1000 μg, more preferably 0.5-500 μg of the ASK1 inhibitor. The pharmaceutical composition may comprise about 0.01-20,000 μg/ml, preferably about 0.1-1000 μg/ml, more preferably 0.5-500 μg/ml, most preferably about 100 μg/ml of the ASK1 inhibitor. The pharmaceutical composition may further comprise a pharmaceutically acceptable carrier or diluent. Carriers and diluents suitable in the pharmaceutical composition are well known in the art.

The pharmaceutical composition may have a pH of about 5.0-10.0, preferably about 5.6-9.0, more preferably about 6.0-8.8, most preferably about 6.5-8.0. For example, the pH may be about 6.2, 6.5, 6.75, 7.0, or 7.5.

The pharmaceutical compositions of the present invention may be formulated for oral, sublingual, intranasal, intraocular, rectal, transdermal, mucosal, topical or parenteral administration. Parenteral administration may include intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids) injection or infusion, preferably intraperitoneal (i.p.) injection in mouse and intravenous (i.v.) in human. Any device suitable for parenteral injection or infusion of drug formulations may be used for such administration. For example, the pharmaceutical composition may be contained in a sterile pre-filled syringe.

The method of the present invention may further comprise administering the subject one or more anti-thrombotic drugs, for example, aspirin and clopidogel. The pharmaceutical composition may further comprise one or more anti-thrombotic drugs, for example, aspirin and clopidogel.

The present invention also provides a method of identifying an inhibitor of an ASK1 protein useful for treating or preventing a thrombotic disease. The method comprises (a) contacting a candidate agent with a test sample comprising the ASK1 protein, and (b) comparing the ASK1 protein activity in the test sample with the ASK1 protein activity in a control sample. A decrease in the ASK1 protein activity in the test sample compared with the control sample indicates that the candidate agent is an ASK1 inhibitor.

The test sample may be a biological sample, comprising, for example, cells, tissues and/or platelets. Preferably, the test sample is obtained from a subject. The subject may have suffered from or may be predisposed to the thrombotic disease. More preferably, the test sample is obtained from a subject who has suffered from the thrombotic disease.

The control sample may be the same as the test sample except it has not been contacted with the candidate agent. For example, the control sample may be the test sample before being contacted with the candidate agent.

In the method of identifying an inhibitor of an AKS1 protein, the ASK1 protein may be a natural protein or recombinant protein. In some embodiments, the ASK1 protein is obtained from activated platelets. The activated platelets may be obtained from a subject who has suffered from a thrombotic disease. The thrombotic disease may be venous thrombosis, arterial thrombosis, atherosclerosis, arthritis, coagulopathy, deep venous thrombosis (DVT), disseminated intravascular coagulopathy (DIC), pulmonary thromboembolism, Budd-Chiari syndrome, Paget-Schroetter diseases, stroke or myocardial infraction. Phosphorylation of a threonine residue in an ASK1 protein corresponding to threonine 838 (T838) of a human full-length ASK1 protein or threonine 845 (T845) of a mouse full-length ASK1 protein may be attenuated in the test sample compared with the control sample. For example, where the ASK1 protein comprises a full-length human ASK1 protein sequence (SEQ ID NO:1), phosphorylation of T838 in SEQ ID NO: 1 may be attenuated in the test sample compared with the control sample. The ASK1 inhibitor identified according to this method may be used in the method of treating or preventing a thrombotic disease in accordance with the present invention.

The present invention further provides a medicament useful for treating or preventing a thrombotic disease in a subject. It comprises an effective amount of an ASK1 inhibitor. It may further comprise one or more anti-thrombotic drugs, for example, aspirin and clopidogel. The thrombotic disease may be selected from the group consisting of venous thrombosis, arterial thrombosis, atherosclerosis, arthritis, coagulopathy, deep venous thrombosis (DVT), disseminated intravascular coagulopathy (DIC), pulmonary thromboembolism, Budd-Chiari syndrome, Paget-Schroetter diseases, stroke and myocardial infraction. The ASK1 protein may be obtained from activated platelets, which may be obtained from a subject who has suffered from the thrombotic disease.

Where the ASK1 protein comprises an amino acid sequence corresponding to the full-length human ASK1 protein (SEQ ID NO: 1) or the full-length mouse ASK1 protein, the ASK1 inhibitor may be capable of attenuate phosphorylation of the threonine residue corresponding to threonine 838 (T838) in the human full-length ASK1 protein or threonine 845 (T845) in the mouse full-length ASK1 protein, respectively. For example, where the ASK1 protein comprises a full-length human ASK1 protein sequence (SEQ ID NO:1), an ASK1 inhibitor in the medicament may attenuate phosphorylation of T838 in SEQ ID NO: 1.

The ASK1 inhibitor in the medicament may be selected from the group consisting of calcium- and integrin-binding protein 1 (CIB1), PP5, 14-3-3ζ, AKT and fragments thereof. Preferably, the ASK1 inhibitor is CIB1 or a fragment thereof. More preferably, the ASK1 inhibitor is a full-length human CIB1 protein or a fragment thereof comprising amino acid residues 1-100. The ASK1 inhibitor may have been identified in accordance with the identifying method of the present invention.

The medicament may further comprise a pharmaceutically acceptable carrier or diluent. The pH of the medicament may be in the range of about 5.0-10.0, preferably about 5.6-9.0, more preferably about 6.0-8.8, most preferably about 6.5-8.0.

For each medicament of the present invention, a method of preparing the medicament is provided. The preparation method comprises admixing an inhibitor of an ASK1 protein with a pharmaceutically acceptable carrier or diluent. The thrombotic disease may be venous thrombosis, arterial thrombosis, atherosclerosis, arthritis, coagulopathy, deep venous thrombosis (DVT), disseminated intravascular coagulopathy (DIC), pulmonary thromboembolism, Budd-Chiari syndrome, Paget-Schroetter diseases, stroke or myocardial infraction. The ASK1 protein may be obtained from activated platelets, which may be obtained from a subject who has suffered from the thrombotic disease. Where the ASK1 protein comprises an amino acid sequence corresponding to the full-length human ASK1 protein (SEQ ID NO: 1) or the full-length mouse ASK1 protein, the ASK1 inhibitor may be capable of attenuate phosphorylation of the threonine residue corresponding to threonine 838 (T838) in the human full-length ASK1 protein or threonine 845 (T845) in the mouse full-length ASK1 protein, respectively. For example, where the ASK1 protein comprises a full-length human ASK1 protein sequence (SEQ ID NO:1), an ASK1 inhibitor in the medicament may attenuate phosphorylation of T838 in SEQ ID NO: 1. The ASK1 inhibitor may be selected from the group consisting of calcium- and integrin-binding protein 1 (CIB1), PP5, 14-3-3ζ, AKT and fragments thereof. Preferably, the ASK1 inhibitor is CIB1 or a fragment thereof. More preferably, the ASK1 inhibitor is a full-length human CIB1 protein or a fragment thereof comprising amino acid residues 1-100. The ASK1 inhibitor may have been identified in accordance with the identifying method of the present invention. The pH of the medicament may be in the range of about 5.0-10.0, preferably about 5.6-9.0, more preferably about 6.0-8.8, most preferably about 6.5-8.0.

### Example 1

### ASK1 Expression in Platelets

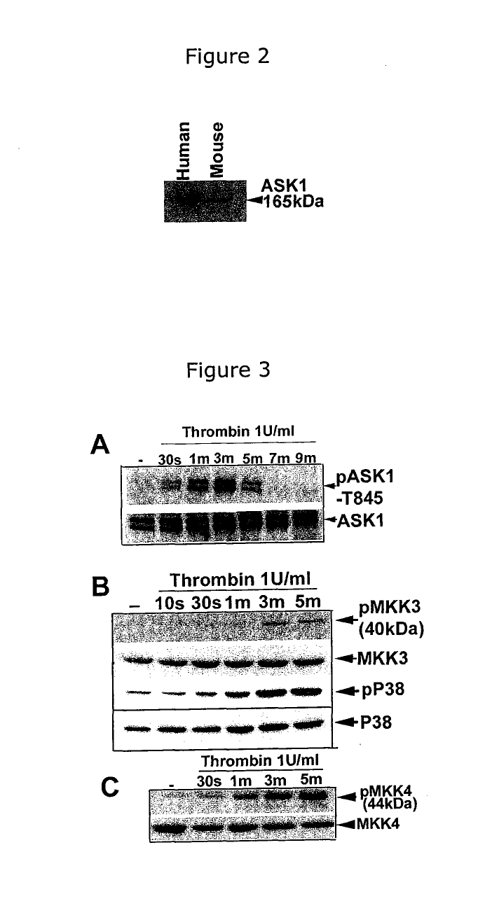

Human platelets were carefully isolated free of red blood cells (RBCs) and white blood cells (WBCs) by differential centrifugation and analyzed for the presence of ASK1 by Western blot analysis using a well-characterized antibody. A substantial amount of ASK1 was expressed in both human and mouse platelets (FIG. 2).

### Example 2

### ASK1 Activation During Agonist-Induced Platelet Stimulation

A study was carried out to determine whether ASK1 was activated during agonist-induced platelet stimulation. A well-characterized antibody specific to the phosphorylated threonine 838 (T838) of ASK1 was used. ASK1 was found not phosphorylated in resting platelets, but was dose-dependently activated by thrombin as indicated by phosphorylation of T838, in the activation loop. Upon stimulation with as low as 0.05 U-1 U/ml of thrombin, ASK1 was activated rapidly and transiently as evidenced by phosphorylation of T838 (FIG. 3A). In a low percentage SDS-PAGE, ASK1 appeared as a doublet.

The effects of ASK1 activation in platelets on downstream substrates were also studied. MAPKK3 and MAPKK4 were expressed in platelets and activated by thrombin. Their activation dependent phosphorylation followed a time course similar to ASK1 activation induced by thrombin (FIGS. 3B and 3C). A robust activation of p38 by thrombin was observed with a time course that followed the activation of ASK1 and MAPKK3/4 (FIG. 3B). These results suggest that G protein-coupled receptor (GPCR)-dependent activation of MAPK cascade is present and functional in platelets.

### Example 3

### Bleeding Phenotype of Ask^{-/-} Mice

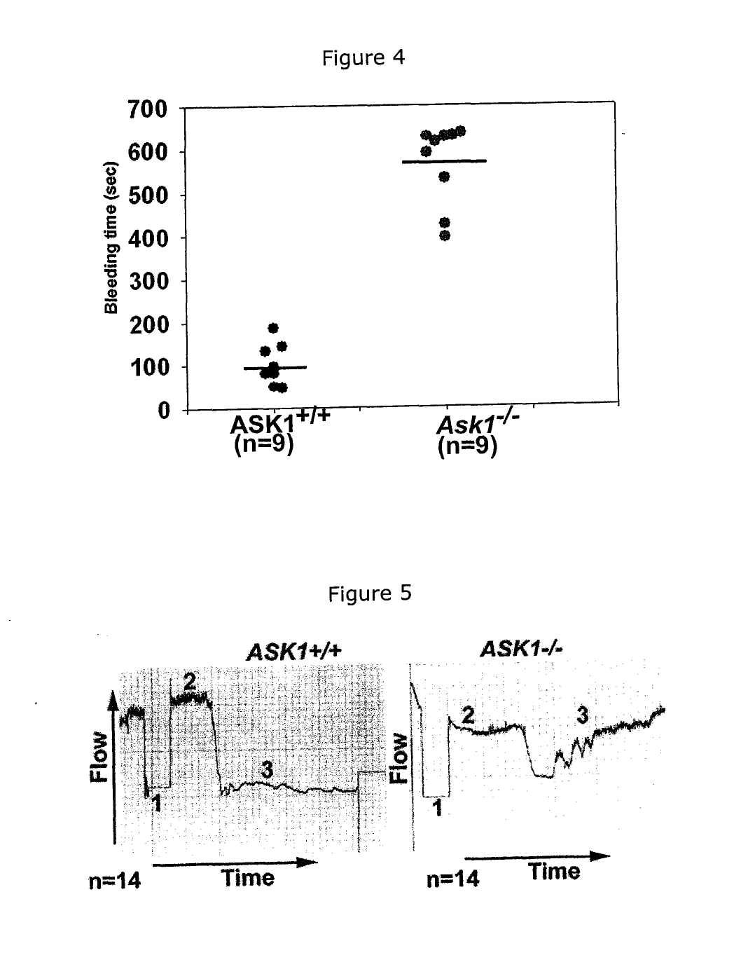

The bleeding phenotype of wild type (WT) mice and Ask1−/− mice of the same genetic background were evaluated by examining the tail bleeding time (FIG. 4). The mean tail bleeding time for WT mice were about 100 s. The Ask1−/− mice had a significantly delayed mean tail bleeding time (576 s), suggesting that Ask1 deficiency results in a severe bleeding phenotype. These results were consistent with the bleeding diathesis observed in older Ask1−/− mice, strongly suggesting that there may be severe defects in thrombotic process in these mice.

### Example 4

### Thrombotic Phenotype of Ask^{-/-} Mice

To further evaluate the thrombotic phenotype of WT and Ask1−/− mice, a 10% FeCl3-induced carotid artery injury was performed as described previously (J. Thromb. Haemost. 2009; 7:1906-14) to observe any difference in time of occlusion or unstable occlusions in Ask1−/− mice compared to WT mice. In this model, FeCl₃ was used to denude endothelial cells, and thus exposed the subendothelial collagen. This represents a well-established model for collagen-dependent thrombosis. Consistent with the finding of greatly extended tail bleeding time in Ask1^{-/-} mice, a WT mouse vessel occluded within 7-9 min whereas it took an Ask1^{-/-} mouse twice that time (˜14 min) to completely occlude a vessel. Furthermore, unlike the WT mouse where the vessel occlusion was stable, in the Ask1−/− mouse the occluded vessel failed to remain occluded indicating unstable thrombus formation (FIG. 5). These results are consistent with the extended tail bleeding time.

### Example 5

### Survival from Occlusive Pulmonary Thromboembolism

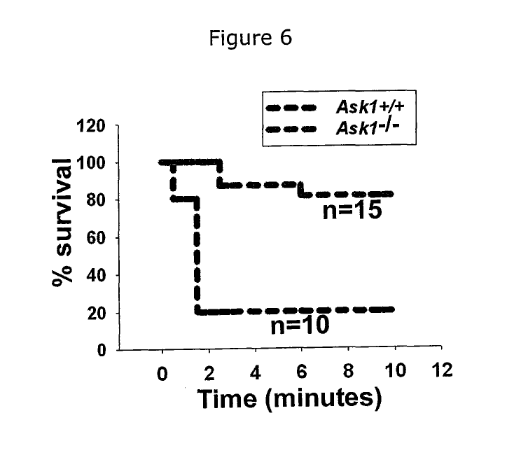

Survival of WT and Ask1−/− mice from occlusive pulmonary thromboembolism was studied. Collagen/epinephrine-induced pulmonary thromboembolism was described previously (Nat. Med. 2001; 7:215-21; Blood 2002; 100:3240-4). In this procedure, a mixture of collagen and epinephrine was injected in the mouse circulation and the survival rate of the mice from occlusive pulmonary thromboembolism was recorded. A marked protection from thromboembolism in Ask1 null mice was observed since significantly more Ask1−/− mice survived compared to WT mice (FIG. 6).

### Example 6

### Platelet Aggregation

Platelet aggregation response was evaluated using WT or Ask1−/− mouse platelets in response to low and high doses of various physiological agonists. Aliquots of platelet rich plasma (PRP) from Ask1^{+/+} and Ask1^{-/-} mice were stimulated in an aggregometer with various concentrations of agonists: 5 nM, 25 nM or 50 nM 2-methylthioadenosine 5′-diphosphate (2MeSADP); 50 μM, 75 μM or 100 μM thrombin/PAR4-peptide (AYPGKF); 0.5 μg/ml, 1 μg/ml or 5 μg/ml collagen; and 0.2 μM, 1 μM or 5 μM thromboxane A₂ (TxA2)-mimetic U46619. Aggregation traces were recorded using Aggrolink software. Platelet aggregation in response to low dose of agonists was blunted in platelets obtained from Ask1−/− mice compared to WT mice. This difference disappeared when a high dose of agonists was used.

### Example 7

### Granular Secretion

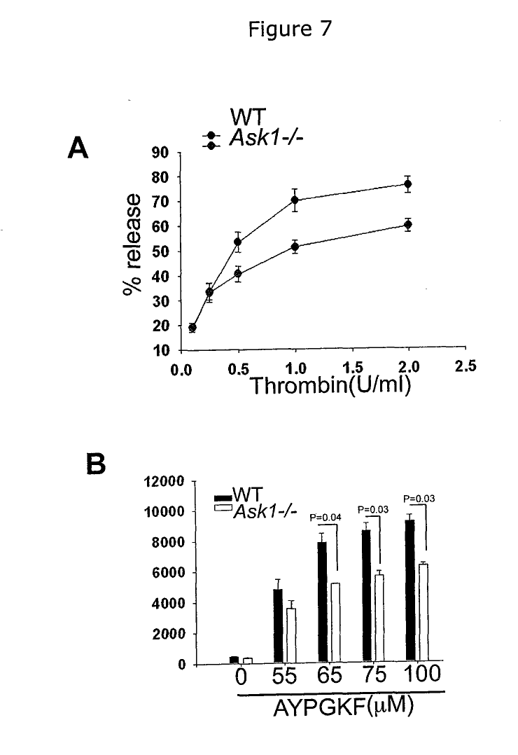

Granular secretion was assessed using WT or Ask1−/− mouse platelets in response to low and high dose of agonists such as thrombin (FIG. 7A) and thrombin/PAR4-peptide (AYPGKF) (FIG. 7B). Dense granule secretion was analyzed by measuring ¹⁴C-serotonin release (FIG. 7A). α-granule secretion was assessed by P-selectin exposure using flow cytometry (FIG. 7B). Ask1 regulated a significant portion of dense granule secretion induced by high dose of thrombin (FIG. 7A). Similar results were obtained when P-selectin exposure was assessed as a measure of α-granule secretion (FIG. 7B). In WT platelets, aspirin pretreatment reduced the amount of serotonin released to that of Ask1−/− platelets, but aspirin had no effect on amount of serotonin released in Ask1−/− platelets. These results suggest that Ask1 regulates a distinct portion of granular secretion, which is entirely dependent on TxA2 generation.

### Example 8

### Activation of Integrin α_{IIb}β₃

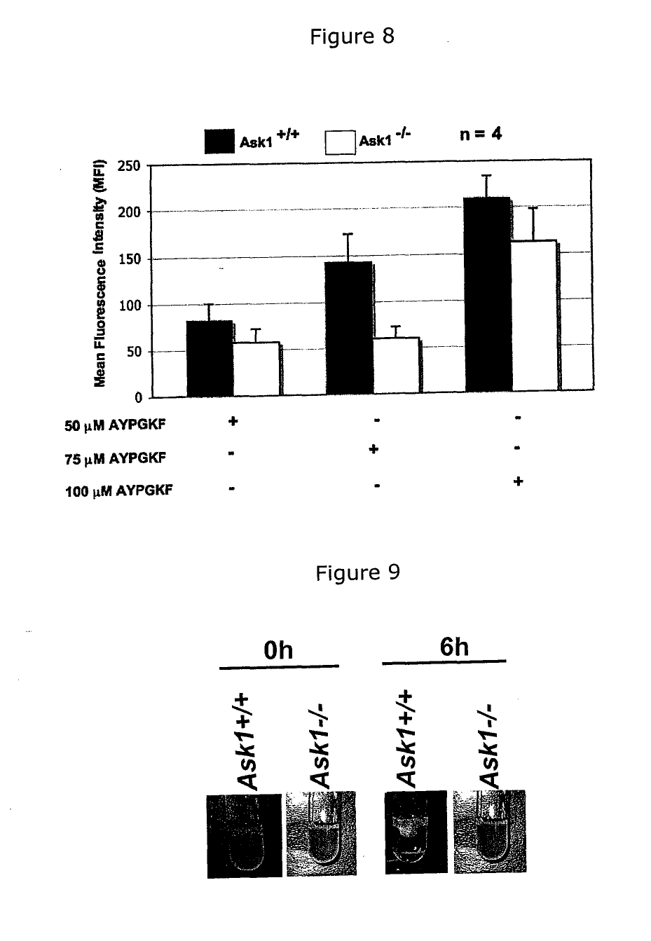

The final step of agonist-induced platelet stimulation is activation of integrin α_{IIb}β₃. Activation of integrin α_{IIb}β₃ was assessed using WT or Ask1−/− mouse platelets activated by 50, 75 or 100 μM PAR4 peptide (AYPGKF). Mean fluorescence intensity (MFI) was measured for FITC-labeled Fg-binding to activated WT or Ask1−/− mouse platelets. A low dose of PAR4 peptide-induced integrin α_{IIb}β₃ activation was attenuated in Ask1−/− mouse platelets (FIG. 8).

### Example 9

### Clot Retraction

Fibrinogen binding to the activated integrin α_{IIb}β₃ induces a cascade of signaling events, termed outside-in signaling, that results in platelet spreading and clot retraction. Clot retraction in WT or Ask1−/− mouse platelets was evaluated. WT platelets retracted clot normally within 90 minutes, whereas Ask1−/− platelets failed to retract clot even after 6-12 hours (FIG. 9). Accordingly, clot retraction was impaired in Ask1 null platelets.

### Example 10

### Phosphatidylserine (PS) Exposure

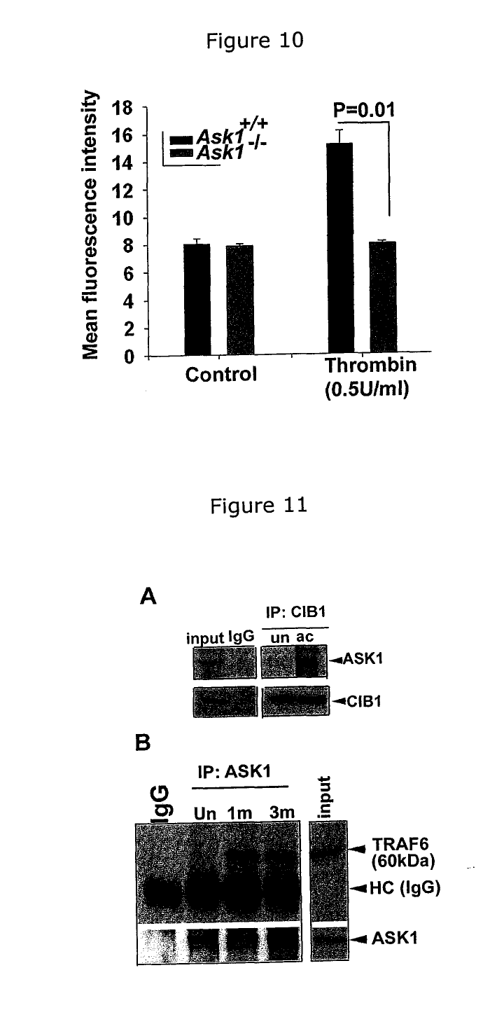

Agonist-induced activation of platelets results in enhanced thrombin generation as a result of exposure of phosphatidylserine (PS), which provides procoagulatory surface. Similar exposure of PS occurs in apoptotic cells. A study was carried out to determine whether Ask1 was needed for PS exposure by platelet agonists. PS exposure was assessed by the ability of cells to bind annexin V using flow cytometry. Lack of Ask1 indeed attenuated exposure of PS-induced by thrombin (FIG. 10).

### Example 11

### Association of ASK1 with CIB1 and TRAF6

Association of ASK1 with CIB1 and TRAF6 was evaluated by co-immunoprecipitation using resting and agonist activated human platelets. An anti-CIB1 antibody was used to immunoprecipitate unstimulated or thrombin stimulated platelet lysates, and Western blot of the immunoprecipitates with an anti-ASK1 antibody showed that CIB1 was associated with ASK1 upon platelet activation (FIG. 11A). An anti-ASK1 antibody was used to immunoprecipitate unstimulated or thrombin stimulated platelet lysates, and Western blot of the immunoprecipitates with an anti-TRAF6 antibody showed that TRAF6 was associated with ASK1 upon platelet activation (FIG. 11B).

### Example 12

### Activation of ASK1 by Oxidized LDL

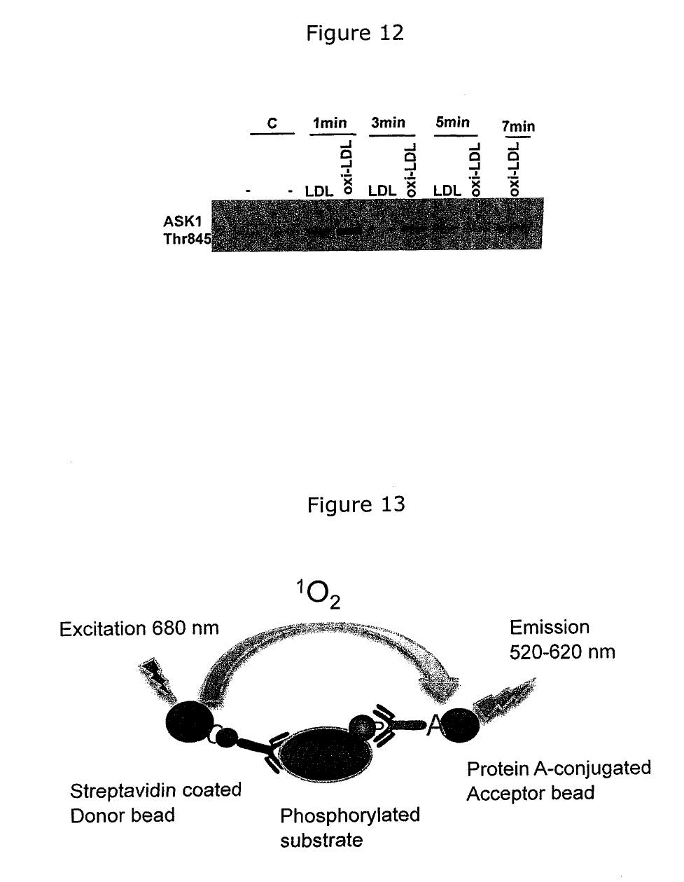

Activation of ASK1 by LDL or oxidized LDL was assessed. Lysates of platelets isolated from healthy donors were exposed to native (LDL) and oxidized LDL for 1, 3, 5 or 7 min, and analyzed by Western blot using an anti-phospho-ASK1 antibody (FIG. 12). Oxidized LDL rapidly activates phosphorylation of ASK1 T838, indicating rapid activation of ASK1.

### Example 13

### Screening Useful ASK1 Inhibitors for Treating or Preventing a Thrombotic Disease

ASK1 inhibitors will be screened and identified from a library of small molecules using human platelets comprising an ASK1 protein. ASK1 protein activity will be assessed based on phosphorylation of the ASK1 protein. A commercially available kit such as an AlphaScreen® SureFire® kit may be used to determine phosphorylation of the ASK1 protein.

For each AlphaScreen® SureFire® kit, the phosphorylated protein is detected using sandwiching antibodies. One antibody is directed against a specific phospho-epitope on the analyte, while the other antibody is directed against another, non-phosphorylated, epitope on a distal part of the analyte. Cells are treated with agents known or suspected to trigger or inhibit phosphorylation of the analyte and then lysed. The lysate is then mixed with the antibodies, the AlphaScreen Donor beads, and the AlphaScreen Acceptor beads. In some kits, the Donor bead associates with the “total” analyte antibody, while the Acceptor bead associates with the anti-phospho specific antibody. Other kits have the reverse configuration, where the anti-phospho antibody associates with the Donor bead, and the “total” antibody associates with the Acceptor bead. If the analyte is phosphorylated, the Donor and Acceptor beads will be brought together. Upon laser excitation at 680 nm, the Donor bead can transfer energy to the Acceptor bead if sufficiently close, resulting in the emission of light at 520-620 nm (FIG. 13). Analyte phosphorylation is detected by an increase in the signal at 520-620 nm.

The assay will use human platelets. In brief, human platelets will be incubated in a buffer and activated by addition of thrombin or exposure to immobilized fibrinogen in a 96 well dish. Activated platelets will be lysed using lysis buffer and incubated with anti-phospho p38 specific antibody, or anti-phospho MEKK6 or anti-phospho MEKK4 or anti-phospho ASK1 antibody and corresponding non-phospho specific antibodies. Anti-phospho MAPKAP2 or anti-phospho HSP27 and corresponding non-phospho specific antibodies will be used. A combination of anti-ASK1 and anti-TRAF6 antibodies will be used to screen a library of small molecules. A candidate small molecule will be in contact with the human platelet lysates comprising an ASK1 protein. The ASK1 protein activity will be determined based on phosphorylation of the ASK1 protein in the contacted platelet lysate, and compared with that in a platelet lysate that has not been contacted with the candidate small molecule. A candidate small molecule that inhibits the ASK1 protein activity, or ASK1 phosphorylation, will be identified as an ASK1 inhibitor useful for treating or preventing a thrombotic disease.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method of treating or preventing a thrombotic disease in a subject in need thereof, comprising administering to the subject an effective amount of a pharmaceutical composition comprising an inhibitor of an apoptosis signal regulating kinase 1 (ASK1) protein.

2. The method of claim 1, wherein the thrombotic disease is selected from the group consisting of venous thrombosis, arterial thrombosis, atherosclerosis, arthritis, coagulopathy, deep venous thrombosis (DVT), disseminated intravascular coagulopathy (DIC), pulmonary thromboembolism, Budd-Chiari syndrome, Paget-Schroetter diseases, stroke and myocardial infraction.

3. The method of claim 1, wherein the ASK1 protein is obtained from activated platelets.

4. The method of claim 3, wherein the activated platelets are obtained from a subject who has suffered from the thrombotic disease.

5. The method of claim 1, wherein the ASK1 protein comprises an amino acid sequence of SEQ ID NO: 1, and wherein the ASK1 inhibitor is capable of attenuating phosphorylation of threonine 838 (T838) in SEQ ID NO: 1.

6. The method of claim 1, wherein the ASK1 inhibitor is selected from the group consisting of calcium- and integrin-binding protein 1 (CIB1), PPS, 14-3-3ζ, AKT and fragments thereof.

7. The method of claim 1, wherein the pharmaceutical composition further comprises a pharmaceutically acceptable carrier or diluent.

8. The method of claim 1, wherein the pharmaceutical composition has a pH of 5.0-10.0.

9. A method of identifying an inhibitor of an apoptosis signal regulating kinase 1 (ASK1) protein useful for treating or preventing a thrombotic disease, comprising
(a) contacting a candidate agent with a test sample comprising the ASK1 protein, and
(b) comparing the ASK1 protein activity in the test sample with the ASK1 protein activity in a control sample that has not been contacted with the candidate agent, whereby a decrease in the ASK1 protein activity in the test sample compared with the control sample indicates that the candidate agent is an ASK1 inhibitor.

10. The method of claim 9, wherein the ASK1 protein is obtained from activated platelets.

11. The method of claim 10, wherein the activated platelets are obtained from a subject who has suffered from the thrombotic disease.

12. The method of claim 11, wherein the thrombotic disease is selected from the group consisting of venous thrombosis, arterial thrombosis, atherosclerosis, arthritis, coagulopathy, deep venous thrombosis (DVT), disseminated intravascular coagulopathy (DIC), pulmonary thromboembolism, Budd-Chiari syndrome, Paget-Schroetter diseases, stroke and myocardial infraction.

13. The method of claim 9, wherein the ASK1 protein comprises an amino acid sequence of SEQ ID NO: 1, and wherein phosphorylation of threonine 838 (T838) in SEQ ID NO: 1 is attenuated in the test sample compared with the control sample.

14. The method of claim 1, wherein the ASK1 inhibitor is identified by a method comprising:
(a) contacting a candidate agent with a test sample comprising the ASK1 protein, and
(b) comparing the ASK1 protein activity in the test sample with the ASK1 protein activity in a control sample that has not been contacted with the candidate agent, whereby a decrease in the ASK1 protein activity in the test sample compared with the control sample indicates that the candidate agent is an ASK1 inhibitor.

15-23. (canceled)

24. A pharmaceutical composition for treating or preventing a thrombotic disease in a subject, comprising an effective amount of an inhibitor of an apoptosis signal regulating kinase 1 (ASK1) protein.

25. The pharmaceutical composition of claim 24, wherein the thrombotic disease is selected from the group consisting of venous thrombosis, arterial thrombosis, atherosclerosis, arthritis, coagulopathy, deep venous thrombosis (DVT), disseminated intravascular coagulopathy (DIC), pulmonary thromboembolism, Budd-Chiari syndrome, Paget-Schroetter diseases, stroke and myocardial infraction.

26. The pharmaceutical composition of claim 24, wherein the ASK1 protein is obtained from activated platelets.

27. The pharmaceutical composition of claim 26, wherein the activated platelets are obtained from a subject who has suffered from the thrombotic disease.

28. The pharmaceutical composition of claim 24, wherein the ASK1 protein comprises an amino acid sequence of SEQ ID NO: 1, and wherein the ASK1 inhibitor is capable of attenuating phosphorylation of threonine 838 (T838) in SEQ ID NO: 1.

29. The pharmaceutical composition of claim 24, wherein the ASK1 inhibitor is selected from the group consisting of calcium- and integrin-binding protein 1 (CIB1), PP5, 14-3-3ζ, AKT and fragments thereof.

30-40. (canceled)
